(19) United States
(12) Patent Application Publication
(10) Pub. No.: US 2022/0172086 A1
(43) Pub. Date: Jun. 2, 2022

(54) SYSTEM AND METHOD FOR PROVIDING UNSUPERVISED MODEL HEALTH MONITORING

(71) Applicant: NICE LTD., Ra'anana (IL)

(72) Inventors: Natan KATZ, Tel Aviv (IL); Gennadi LEMBERSKY, Haifa (IL)

(73) Assignee: NICE LTD., Ra'anana (IL)

(21) Appl. No.: 17/106,293

(22) Filed: Nov. 30, 2020

(51) Int. Cl. G06N 7/00; G06N 20/00

(52) U.S. Cl. CPC G06N 7/005; G06N 20/00

(57) ABSTRACT

Systems and methods for providing unsupervised model health monitoring extract from an interaction database, first and second random samples of interaction data relating to first and second sets of interactions during first and second periods of time; score each interaction of the first and second sets of interactions by applying a predictive model to the related interaction data to produce first and second sets of interaction scores; identify a plurality of sub-populations among the first and second sets of interaction scores by applying a clustering model to the first and second sets of interaction scores; measure distances between each of the plurality of sub-populations among the first and second sets of interaction scores; compare the distances of the first period of time and the distances of the second period of time; and generate an alert when the comparison exceeds a predefined threshold.

### FIELD OF THE INVENTION

The present invention is in the field of model health monitoring. In particular, the present invention is directed systems and methods providing unsupervised model health monitoring.

### BACKGROUND OF THE INVENTION

Model health monitoring is an important component of the predictive analytics life cycle. It is used to identify when the performance of a predictive model deteriorates due, for example, to content shift or some other reason. A model health monitoring component periodically assesses the performance of a model on a new data sample by evaluating it against true labels. If, however, there are no relevant labels, the performance of a model cannot be evaluated. Current tools and metrics are unable to give a rough estimate of a model's performance in an unsupervised manner. Furthermore, current tools and metrics are unable to provide an estimate that is highly correlated to usual model performance metrics, such as accuracy, recall or PRAUC (an area under a precision-recall curve). What are needed, therefore, are systems and methods which evaluate each interaction in a series or chain of interactions while taking into account the evolution of the customer experience over the chain.

### SUMMARY OF EMBODIMENTS OF THE INVENTION

Various embodiments of the invention include systems and methods for providing unsupervised model health monitoring. Embodiments of the invention are performed on a computer having a processor, a memory, and one or more code sets stored in the memory and executed by the processor. In various embodiments of the invention, systems and methods may include extracting, by the processor, from an interaction database, a first random sample of interaction data relating to a first set of interactions during a first period of time; scoring, by the processor, each interaction of the first set of interactions by applying a predictive model to the related interaction data to produce a first set of interaction scores; identifying, by the processor, a plurality of sub-populations among the first set of interaction scores by applying a clustering model to the first set of interaction scores; measuring, by the processor, a distance between each of the plurality of sub-populations among the first set of interaction scores; extracting, by the processor, from the interaction database, a second random sample of interaction data relating to a second set of interactions during a second period of time; scoring, by the processor, each interaction of the second set of interactions by applying the predictive model to the related interaction data to produce a second set of interaction scores; identifying, by the processor, a plurality of sub-populations among the second set of interaction scores by applying the clustering model to the second set of interaction scores; measuring, by the processor, a distance between each of the plurality of sub-populations among the second set of interaction scores; comparing, by the processor, the distances of the first period of time and the distances of the second period of time; and generating, by the processor, an alert when the comparison exceeds a predefined threshold.

In some embodiments, the clustering model is a gaussian mixture model; and the sub-populations are gaussians. In some embodiments, the measured distance is the distance between means of the gaussians. In some embodiments, the measured distance is a KL-divergence between a lower gaussian and a upper gaussian; and a mean of each gaussian is used to identify the lower gaussian and the upper gaussian. In some embodiments, the predictive model is one of an SVM-based model, a deep learning model, a neural network-based model, a logistic regression model, a linear regression model, a nearest neighbor model, a decision tree model, a PCA-based model, a naive Bayes classifier model, and a k-means clustering model.

In some embodiments, each interaction is represented by a unique identifier; and a plurality of unique identifiers is randomly selected when extracting the random samples. In some embodiments, an interaction comprises data relating to an interaction between a customer and call center in a communication channel. In some embodiments, a communication channel is one of automatically transcribed content of a voice conversation, e-mail, chat, and text message.

In various embodiments of the invention, systems and methods for providing unsupervised model health monitoring may include a computer having a processor and a memory, and one or more code sets stored in the memory and executed by the processor, which, when executed, configured the processor to: extract from an interaction database, a first random sample of interaction data relating to a first set of interactions during a first period of time; score each interaction of the first set of interactions by applying a predictive model to the related interaction data to produce a first set of interaction scores; identify a plurality of sub-populations among the first set of interaction scores by applying a clustering model to the first set of interaction scores; measure a distance between each of the plurality of sub-populations among the first set of interaction scores; extract from the interaction database, a second random sample of interaction data relating to a second set of interactions; score each interaction of the second set of interactions by applying the predictive model to the related interaction data to produce a second set of interaction scores; identify a plurality of sub-populations among the second set of interaction scores by applying the clustering model to the second set of interaction scores; measure a distance between each of the plurality of sub-populations among the second set of interaction scores; compare the distances of the first period of time and the distances of the second period of time; and generate an alert when the comparison exceeds a predefined threshold.

In various embodiments of the invention, systems and methods for providing unsupervised model health monitoring may include extracting, by the processor, from an interaction database, a plurality of random samples of interaction data relating to respective sets of interactions during a plurality of periods of time; scoring, by the processor, each interaction of each respective set of interactions by applying a predictive model to the related interaction data to produce a set of interaction scores for each respective set; identifying, by the processor, a plurality of sub-populations among the each respective set of interaction scores by applying a clustering model to each respective set of interaction scores; measuring, by the processor, a respective distance between each of the plurality of sub-populations among each of the respective sets of interaction scores; comparing, by the processor, the respective distances of each period of time; and generating, by the processor, an alert when a comparison exceeds a predefined threshold for at least one of the respective comparisons.

These and other aspects, features and advantages will be understood with reference to the following description of certain embodiments of the invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 shows a high-level diagram illustrating an exemplary configuration of a system 100 for performing one or more aspects of the invention;

FIG. 2 is a high-level diagram illustrating an example configuration of a method workflow 200 for providing unsupervised model health monitory, according to at least one embodiment of the invention;

FIG. 3 shows an example plot of a scores distribution is shown in graph 300 according to at least one embodiment of the invention; and

FIG. 4 shows an example plot of two gaussians is shown in graph 400 according to at least one embodiment of the invention.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF EMBODIMENTS OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory processor-readable storage medium that may store instructions, which when executed by the processor, cause the processor to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof may occur or be performed simultaneously, at the same point in time, or concurrently.

Embodiments of the invention may provide systems and methods providing unsupervised model health monitoring. Embodiments of the invention may provide an estimate of predictive model performance on a new unlabeled dataset (i.e., an unsupervised dataset). Intuitively, any binary predictive model tries to split the data into two populations (e.g., a positive class and a negative class). The more accurate the model is, the better it separates two populations. Therefore, by measuring the degree of separation between two populations, embodiments of the invention may provide an estimate of a model's accuracy.

The following describes a specific example implementation according to various embodiments of the invention. Suppose one needs to compare the Q1 2020 (i.e., first quarter of the year 2020) performance of a predictive model that predicts a negative/positive outcome of an interaction to the performance of the same predictive model in Q2 2020 (i.e., second quarter of the year 2020), in order to assess a possible impact due to the COVID-19 pandemic. Embodiments of the invention may address this problem by, for example, applying the following steps: 1. Extract a random sample from the Q1 data and use the predictive model to score each sample in the extracted dataset. 2. Use a clustering algorithm to split the resulting list of scores into two clusters. For example, a Gaussian Mixture Model (GMM) may be used to create two gaussians. 3. Compute a metric that measures a distance between the two clusters. One such exemplary metric may be a distance between means of two gaussians. Another such exemplary metric may be a KL-divergence between lower (e.g., negative) and upper (e.g., positive) gaussians, using a mean of each gaussian to identify the upper gaussian and a lower gaussian. 4. Repeat steps 1-3 on the Q2 2020 data. 5. Compare the metrics for the Q1 and Q2 datasets. If the Q2 metric is significantly different from the Q1 metric (e.g., 20% lower), send an alert to a user.

As another example, a binary classifier may differentiate “bad” conversations from “good” ones, in which a lower (e.g., negative) gaussian may be associated with “bad” calls and an upper (e.g., positive) gaussian may be associated with “good” calls. Commonly, “lower” refers to a gaussian closer to 0 or to the left (on a graph) and “upper” refers to a gaussian closer to 1 or to the right (on the graph).

It should be clear to those skilled in the art that embodiments of the invention described herein are model agnostic, i.e., working with any type of predictive model, e.g., Support-vector machines (SVMs), Artificial neural networks (ANNs), Convolutional Neural Networks (CNNs), logistic regression, linear regression, nearest neighbor model, decision tree model, PCA-based model, naive Bayes classifier model, k-means clustering, or other classical machine learning models, deep learning models, or neural network-based models, etc. Furthermore, in situations where there is no labeled data, in prior art systems the only option for performance monitoring of predictive models is to manually label, for example, a few thousand examples or more, which is expensive, time-consuming, and subjective (i.e., inaccurate). Embodiments of the invention, however, support the processing of unlabeled data, significantly improving processing efficiency while significantly reducing storage requirements, as storage is not required to be allocated to labels.

Embodiments of the invention eliminate the need for labeled data for performance monitoring of predictive models. This is based on a non-trivial understanding of how a change in predictive model performance is reflected through the distribution of its scores. Embodiments of the invention create a “representation” of a predictive model by clustering predictive scores over a random sample of interactions using a clustering algorithm, for example GMM. This representation is essentially a set of cluster parameters. Such a representation may be extracted using, for example, random samples from different time periods. In various embodiments, several metrics may be computed through a comparison of model representation between two periods of time. These metrics are highly correlated to standard model performance metrics (e.g., PRAUC) and therefore may indicate if there was a significant change (e.g., drop) in a model behavior over time (e.g., day to day, month to month, quarter to quarter, year to year, or any other defined period of time, etc.).

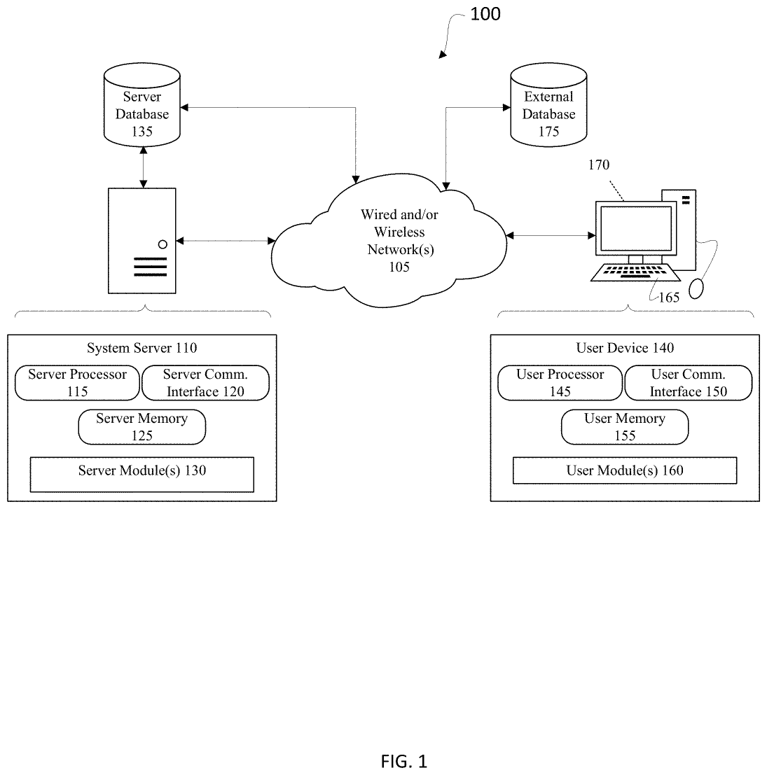

These and other features of embodiments of the invention will be further understood with reference to FIGS. 1-4 as described herein.

FIG. 1 shows a high-level diagram illustrating an exemplary configuration of a system 100 for performing one or more aspects of the invention described herein, according to at least one embodiment of the invention. System 100 includes network 105, which may include the Internet, one or more telephony networks, one or more network segments including local area networks (LAN) and wide area networks (WAN), one or more wireless networks, or a combination thereof. System 100 also includes a system server 110 constructed in accordance with one or more embodiments of the invention. In some embodiments, system server 110 may be a stand-alone computer system. In other embodiments, system server 110 may include a network of operatively connected computing devices, which communicate over network 105. Therefore, system server 110 may include multiple other processing machines such as computers, and more specifically, stationary devices, mobile devices, terminals, and/or computer servers (collectively, “computing devices”). Communication with these computing devices may be, for example, direct or indirect through further machines that are accessible to the network 105.

System server 110 may be any suitable computing device and/or data processing apparatus capable of communicating with computing devices, other remote devices or computing networks, receiving, transmitting and storing electronic information and processing requests as further described herein. System server 110 is, therefore, intended to represent various forms of digital computers, such as laptops, desktops, workstations, personal digital assistants, servers, blade servers, mainframes, and other appropriate computers and/or networked or cloud-based computing systems capable of employing the systems and methods described herein.

System server 110 may include a server processor 115 which is operatively connected to various hardware and software components that serve to enable operation of the system 100. Server processor 115 serves to execute instructions to perform various operations relating to advanced search, and other functions of embodiments of the invention as described in greater detail herein. Server processor 115 may be one or several processors, a central processing unit (CPU), a graphics processing unit (GPU), a multi-processor core, or any other type of processor, depending on the particular implementation.

System server 110 may be configured to communicate via communication interface 120 with various other devices connected to network 105. For example, communication interface 120 may include but is not limited to, a modem, a Network Interface Card (NIC), an integrated network interface, a radio frequency transmitter/receiver (e.g., Bluetooth wireless connection, cellular, Near-Field Communication (NFC) protocol, a satellite communication transmitter/receiver, an infrared port, a USB connection, and/or any other such interfaces for connecting the system server 110 to other computing devices and/or communication networks such as private networks and the Internet.

In certain implementations, a server memory 125 is accessible by server processor 115, thereby enabling server processor 115 to receive and execute instructions such as a code, stored in the memory and/or storage in the form of one or more software modules 130, each module representing one or more code sets. The software modules 130 may include one or more software programs or applications (collectively referred to as the “server application”) having computer program code or a set of instructions executed partially or entirely in server processor 115 for carrying out operations for aspects of the systems and methods disclosed herein, and may be written in any combination of one or more programming languages. Server processor 115 may be configured to carry out embodiments of the present invention by, for example, executing code or software, and may execute the functionality of the modules as described herein.

In accordance with embodiments of FIG. 1, the exemplary software modules may include a communication module and other modules as described here. The communication module may be executed by server processor 115 to facilitate communication between system server 110 and the various software and hardware components of system 100, such as, for example, server database 135, client device 140, and/or external database 175 as described herein.

Of course, in some embodiments, server modules 130 may include more or less actual modules which may be executed to enable these and other functionalities of the invention. The modules described herein are, therefore, intended to be representative of the various functionalities of system server 110 in accordance with some embodiments of the invention. It should be noted that, in accordance with various embodiments of the invention, server modules 130 may be executed entirely on system server 110 as a stand-alone software package, partly on system server 110 and partly on user device 140, or entirely on user device 140.

Server memory 125 may be, for example, random access memory (RAM) or any other suitable volatile or non-volatile computer readable storage medium. Server memory 125 may also include storage which may take various forms, depending on the particular implementation. For example, the storage may contain one or more components or devices such as a hard drive, a flash memory, a rewritable optical disk, a rewritable magnetic tape, or some combination of the above. In addition, the memory and/or storage may be fixed or removable. In addition, memory and/or storage may be local to the system server 110 or located remotely.

In accordance with further embodiments of the invention, system server 110 may be connected to one or more database(s) 135, for example, directly or remotely via network 105. Database 135 may include any of the memory configurations as described herein, and may be in direct or indirect communication with system server 110. In some embodiments, database 135 may store information relating to user documents. In some embodiments, database 135 may store information related to one or more aspects of the invention.

As described herein, among the computing devices on or connected to the network 105 may be one or more user devices 140. User device 10 may be any standard computing device. As understood herein, in accordance with one or more embodiments, a computing device may be a stationary computing device, such as a desktop computer, kiosk and/or other machine, each of which generally has one or more processors, such as user processor 145, configured to execute code to implement a variety of functions, a computer-readable memory, such as user memory 155, a user communication interface 150, for connecting to the network 105, one or more user modules, such as user module 160, one or more input devices, such as input devices 165, and one or more output devices, such as output devices 170. Typical input devices, such as, for example, input devices 165, may include a keyboard, pointing device (e.g., mouse or digitized stylus), a web-camera, and/or a touch-sensitive display, etc. Typical output devices, such as, for example output device 170 may include one or more of a monitor, display, speaker, printer, etc.

In some embodiments, user module 160 may be executed by user processor 145 to provide the various functionalities of user device 140. In particular, in some embodiments, user module 160 may provide a user interface with which a user of user device 140 may interact, to, among other things, communicate with system server 110.

Additionally or alternatively, a computing device may be a mobile electronic device (“MED”), which is generally understood in the art as having hardware components as in the stationary device described above, and being capable of embodying the systems and/or methods described herein, but which may further include componentry such as wireless communications circuitry, gyroscopes, inertia detection circuits, geolocation circuitry, touch sensitivity, among other sensors. Non-limiting examples of typical MEDs are smartphones, personal digital assistants, tablet computers, and the like, which may communicate over cellular and/or Wi-Fi networks or using a Bluetooth or other communication protocol. Typical input devices associated with conventional MEDs include, keyboards, microphones, accelerometers, touch screens, light meters, digital cameras, and the input jacks that enable attachment of further devices, etc.

In some embodiments, user device 140 may be a “dummy” terminal, by which processing and computing may be performed on system server 110, and information may then be provided to user device 140 via server communication interface 120 for display and/or basic data manipulation. In some embodiments, modules depicted as existing on and/or executing on one device may additionally or alternatively exist on and/or execute on another device. For example, in some embodiments, one or more modules of server module 130, which is depicted in FIG. 1 as existing and executing on system server 110, may additionally or alternatively exist and/or execute on user device 140. Likewise, in some embodiments, one or more modules of user module 160, which is depicted in FIG. 1 as existing and executing on user device 140, may additionally or alternatively exist and/or execute on system server 110.

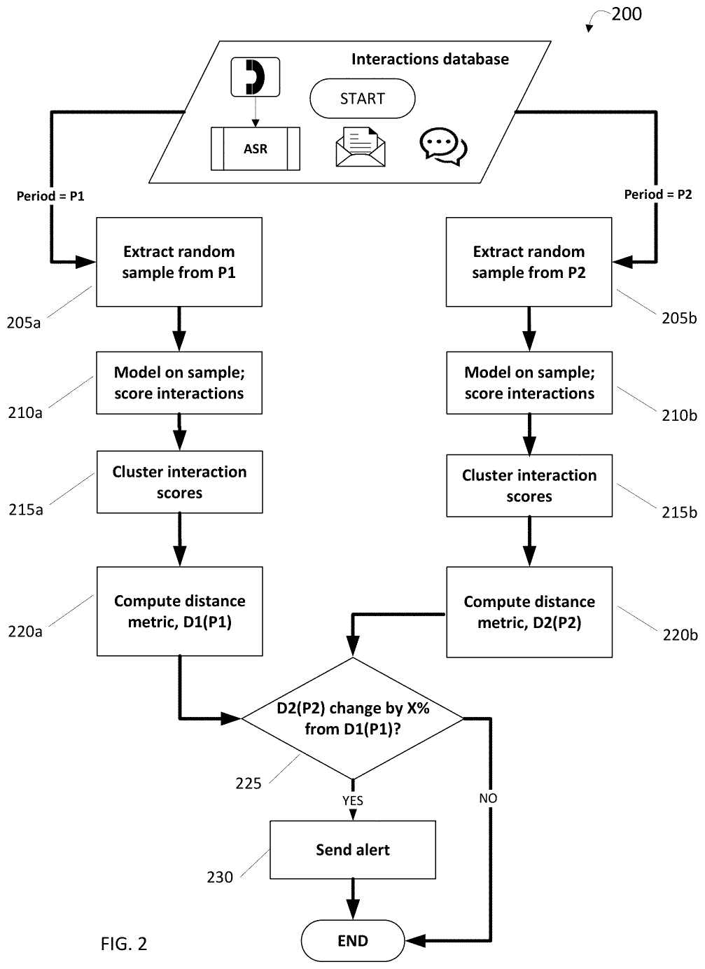

FIG. 2 is a high-level diagram illustrating an example configuration of a method workflow 200 for providing unsupervised model health monitoring, according to at least one embodiment of the invention. In some embodiments, method workflow 200 may be performed on a computer (e.g., system server 110) having a processor (e.g., server processor 115), memory (e.g., server memory 125), and one or more code sets or software (e.g., server module(s) 130) stored in the memory and executing in or executed by the processor. In some embodiments, method workflow 200 begins at step 205a, when the processor is configured to extract from an interaction database, a first random sample of interaction data relating to a first set of interactions during a first period of time. In some embodiments, interaction data may be stored, for example, in an interaction database. In some embodiments, an input may be an interactions dataset comprising interaction data. The dataset may contain data relating to interactions (e.g., between a customer and call center) from one or more channels, such as automatically transcribed content (ASR) of voice conversations, e-mails, chats, text messages, etc. For each interaction, the database may contain at least the following information: ID (Number)—a unique identifier of an interaction; and filename (String)—a path to a file that contains the content (text) of the interaction. Of course, other data relating to an interaction may also or alternatively be included in the interaction data and stored in the interaction database.

In some embodiments, the processor may be configured to extract a random set of interactions from a given period (denoted P1 herein), for example, Q1 2020, or any other predefined time period. One of a variety of random sampling algorithms may be used in accordance with embodiments of the invention. For example, a unique identifier of an interaction may be used to decide if a particular interaction should be selected or not. In some embodiments, the set of random samples may contain, for example, 25,000 interactions. Of course, more or less interactions may be used. In some embodiments, the input of step 205a may be a period, e.g., P1, all interactions from the period (P1), and a predefined size of a sample (S); an output may be a list of interaction filenames, i.e., a random sample set of size S.

An example random sample algorithm for executing step 205a is representing in the following pseudocode:
RandomSample(P, S)
   Interactions=[ ]//empty list
   p_set=ALL INTERACTIONS from P from interactions dataset
   total=SIZE(P_set)
   mod=FLOOR(total/sample_size)//FLOOR rounds the real number to the nearest smaller integer
   for each interaction in P_set:
      if hash(interaction.ID) % mod==0://mod refers to a portion of data that can be extracted, e.g., 100 from 1000 is 10%=>mod=10 interactions.add(interaction.filename)
   return interactions

At step 205b, in some embodiments, the processor may extract from the interaction database, a second random sample of interaction data relating to a second set of interactions. In some embodiments, step 205b mirrors that of step 205a, and the process may be essentially the same or similar. In some embodiments, an input may be a second interactions dataset comprising interaction data, e.g., as described herein. The dataset may contain data relating to interactions from one or more channels. In some embodiments, the processor may be configured to extract a random set of interactions from a second given period (denoted P2 herein), for example, Q2 2020, or any other predefined time period that follows or precedes the initial period. One of a variety of random sampling algorithms may be used in accordance with embodiments of the invention, for example, as described herein.

In some embodiments, the input of step 205b may be a second period, e.g., P2, all interactions from the second period (P2), and a predefined size of a sample (S), typically the same size as in step 205a; an output may be a list of interaction filenames, i.e., a random sample set of size S from P2.

At step 210a, in some embodiments, the processor may be configured to score each interaction of the first set of interactions by applying a predictive model to the related interaction data to produce a first set of interaction scores. Specifically, the processor may be configured to apply a predictive model (denoted M herein) whose health is to be tested to the sample set of P1 interaction data (from step 205a) to produce a first set of interaction scores. In some embodiments, the predictive model may be applied to each interaction in the sample set of P1 interaction data (e.g., in an iterative process). Accordingly, in some embodiments, the input may be a list of interaction filenames (output from 205a) and a predictive model M, and the output may be a list or other collection of scores.

An example model-applying algorithm for scoring each interaction in the sample set of P1 interaction data in step 210a is representing in the following pseudocode:
RunModel(M: Model, interactions: list of filenames):
   scores=[ ]//empty list
   for each interaction in interactions:
      scores.add (M.predict(interaction))
   return scores

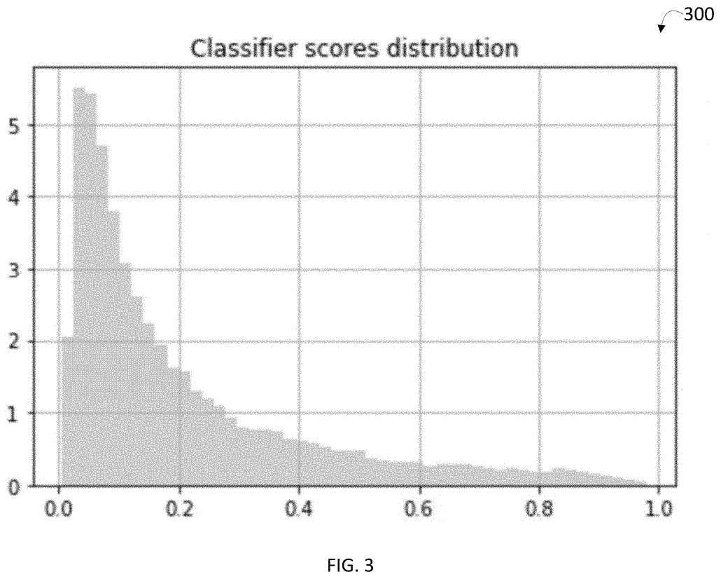

Turning briefly to FIG. 3, an example plot of a scores distribution is shown in graph 300 according to at least one embodiment of the invention.

At step 210b, in some embodiments, the processor may be configured to score each interaction of the second set of interactions by applying the predictive model to the related interaction data to produce a second set of interaction scores. In some embodiments, step 210b mirrors that of step 210a, and the process may be essentially the same or similar. Specifically, the processor may be configured to apply the same predictive model M whose health is to be tested to the sample set of P2 interaction data (from step 205b) to produce a second set of interaction scores. In some embodiments, the predictive model may be applied to each interaction in the sample set of P2 interaction data (e.g., in an iterative process). Accordingly, in some embodiments, the input may be a list of interaction filenames (output from 205b) and the predictive model M, and the output may be a second list or other collection of scores.

At step 215a, in some embodiments, the processor may be configured to identify a first plurality of sub-populations among the first set of interaction scores by applying a clustering model to the first set of interaction scores. For example, in some embodiments, a Gaussian Mixture Model (GMM) may be applied to the first set of interaction scores. A GMM is a clustering algorithm or model that splits a given set of samples into normally distributed subsets (also referred to as gaussians). In some embodiments, a standard GMM model may be applied to the list of scores generated in step 210a. Those skilled in the art will recognize that there are many variations of the GMM algorithm as well as many other clustering models, any of which may be implemented according to various embodiments without deviating from the invention. For example, in various embodiments, other clustering algorithms may be used such as, e.g., Beta-Distribution Mixture Model, K-Means, Density-based spatial clustering of applications with noise (DBSCAN), and others.

In some embodiments, the plurality of resulting components (e.g., gaussians) may be set to, e.g., two (2), such that the output of this phase is two univariate sub-populations (e.g., two gaussian distributions) defined by their mean and variance. Accordingly, in some embodiments, the input may be a list of scores from step 210a and the output may be two gaussians: G1(mean, variance), G2(mean, variance). It should be noted that in various embodiments more than two sub-populations may be used as well.

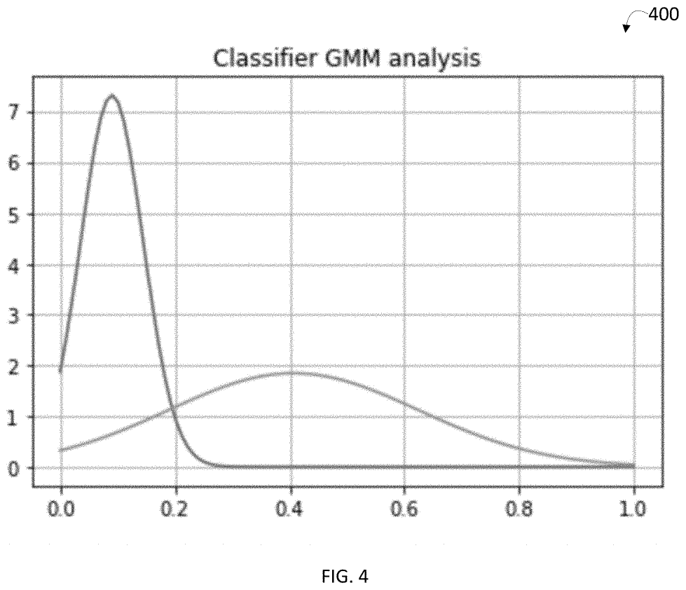

Turning briefly to FIG. 4, an example plot of two gaussians is shown in graph 400 according to at least one embodiment of the invention.

At step 215b, in some embodiments, the processor may be configured to identify a second plurality of sub-populations among the second set of interaction scores by applying a clustering model to the second set of interaction scores. In some embodiments, step 215b mirrors that of step 215a, and the process may be essentially the same or similar. For example, in some embodiments, the same clustering model (e.g., GMM) as in step 215a may be applied to the second set of interaction scores, e.g., in some embodiments, a standard GMM model may be applied to the list of scores generated in step 210b. In some embodiments, the plurality of resulting components (e.g., gaussians) may be set to, e.g., two (2), such that the output of this phase is two univariate sub-populations (e.g., two gaussian distributions) defined by their mean and variance. Accordingly, in some embodiments, the input may be a list of scores from step 210b and the output may be two gaussians: G3(mean, variance), G4(mean, variance). Those skilled in the art will recognize that, in various embodiments, the clustering model and number of sub-populations which are selected in step 215a may be likewise selected in step 215b, such that the results of each step may be compared, as explained in detail herein.

At step 220a, in some embodiments, the processor may be configured to measure a distance (denoted D1) between each of the plurality of sub-populations among the first set of interaction scores. For example, if a GMM algorithm producing two gaussians was applied at step 215a, the processor may compute the distance between the two gaussians, e.g., a distance between the means of the two gaussians, i.e., a distance between the centroids of the two clusters. Those skilled in the art will recognize that different distance metrics may be used, for example, absolute mean difference, KL-divergence, etc. In some embodiments, e.g., when an absolute mean difference is used, the input may be the gaussians from 215a, G1(mean, variance), G2(mean, variance), and the output may be a distance, D1.

At step 220b, in some embodiments, the processor may be configured to measure a distance (D2) between each of the plurality of sub-populations among the second set of interaction scores. For example, if a GMM algorithm producing two gaussians was applied at step 215b, the processor may compute the distance between the two gaussians. In some embodiments, e.g., when an absolute mean difference is used, the input may be the gaussians from 215b, G3(mean, variance), G4(mean, variance), and the output may be a distance, D2.

At step 225, in some embodiments, the processor may compare the distance (D1) of the first period of time (P1) and the distance (D2) of the second period of time (P2), and, at step 230 the processor may generate an alert when the comparison exceeds a predefined threshold, as explained herein. For example, in some embodiments, the processor may compare D1 and D2, and if a distance metric changes by a threshold amount, e.g., drops below a given predefined threshold (denoted “alpha” herein), the processor may by determine that an alert should be generated. Accordingly, in some embodiments, an input may be two distance metrics: D1(P1) and D2(P2) and a threshold, alpha, and an output may be an alert flag (or a determination that no alert is required) at this time. It should be noted that various comparisons may be provided between D1 and D2, as well as various thresholds.

An example distance-comparison algorithm is representing in the following pseudocode:
CompareDistances(D1, D2, alpha):
   return D2/D1<(1−alpha)

An example comparison according to an embodiment of the invention is as follows:

$$D 1 = 0.24$$

$$D 2 = 0.20$$

$$Alpha = 0.2 // 20 \%$$

$$\begin{matrix}Flag = D 2 / D 1 < ( 1 - alpha ) \\ = 0.2 / 0.24 < ( 1 - 0.2 ) \\ = 0.833 < 0.8 \\ = False // no alert\end{matrix}$$

In this example, because the comparison between D1 and D2 did not drop below the threshold, alpha, the model is assumed to be in good health (as defined by the threshold and the various other parameters) no alert will be sent. On the other hand, should the comparison reach or exceed the predefined threshold (e.g., dropping below the threshold), in some embodiments, the processor may be configured to sends an alert. In various embodiments, an alert implementation may depend on the application or policy and may be one or more of a log message, an on-screen message, an e-mail, SMS, or other communication, e.g., to an administrator.

Embodiments of the invention provide significant improvements over currently available systems and methods. For example, embodiments of the invention enable users to identify models (e.g., predictive model) which are displaying poor health (e.g., are no longer providing predictions within a predefined threshold of prior predictions). Furthermore, embodiments of the invention may be integrated in a ‘Model Health’ service that is invoked, e.g., periodically to monitor performance of various predictive models. The service may be connected to an Interactions database, e.g., to extract needed interactions and metadata. In some embodiments, the processor may also invoke a ‘Predictive decision engine’ component to run a model on a set of selected interactions, as described herein. In case an alert should be generated, an ‘alert’ service may be invoked, as described herein.

Unless explicitly stated, the method embodiments described herein are not constrained to a particular order or sequence. For example, while the steps of method workflow 200 with respect to P1 and P2 are described herein in one embodiment as following a back-and-forth order (i.e., 205a→205b→210a→210b→215a→215b→220a→220b), in other embodiments different orders of operations may be executed. For example, in some embodiments, the analysis of the sample of P1 may occur prior to the analysis of the sample of P2 (i.e., 205a→210a→215a→220a→205b→210b→215b→220b), or both analyses may occur contemporaneously (i.e., [205a,205b]→[210a,210b]→[215a,215b]→[220a,220b]), etc.

Furthermore, all formulas and pseudocode described herein are intended as examples only and other or different formulas may be used. Additionally, some of the described method embodiments or elements thereof may occur or be performed at the same point in time.

Additionally, the following chart provides definitions for various variables described herein according to embodiments of the invention:

| Variable | Type | Description |
|---|---|---|
| ID | STRING | Unique identifier of an Interaction |
| Filename | STRING | A full path to the interaction content |
| Period | STRUCT: | A specified period of time for |
| | DATE: start | comparison |
| | DATE: end | |
| Sample_size | INTEGER | A size of sample. Default value, e.g., |
| | | 25,000 |
| Interactions | LIST<STRING> | List of interaction filenames |
| Model | MODEL | A complex object that implements a |
| | | predictive model |
| Scores | LIST<FLOAT> | List of scores, each score is a float |
| | | number |
| Gaussian | STRUCT: | |
| | FLOAT: mean | |
| | FLOAT: variance | |
| Distance | FLOAT | A positive measure of a distance |
| | | between gaussians |
| Alpha | FLOAT | A threshold that defines if an alert |
| | | should be generated |
| Flag | BOOLEAN | If True, generate an alert |

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

What is claimed is:

1. A method for providing unsupervised model health monitoring, performed on a computer having a processor, a memory, and one or more code sets stored in the memory and executed by the processor, the method comprising:
extracting, by the processor, from an interaction database, a first random sample of interaction data relating to a first set of interactions during a first period of time;
scoring, by the processor, each interaction of the first set of interactions by applying a predictive model to the related interaction data to produce a first set of interaction scores;
identifying, by the processor, a plurality of sub-populations among the first set of interaction scores by applying a clustering model to the first set of interaction scores;
measuring, by the processor, a distance between each of the plurality of sub-populations among the first set of interaction scores;
extracting, by the processor, from the interaction database, a second random sample of interaction data relating to a second set of interactions during a second period of time;
scoring, by the processor, each interaction of the second set of interactions by applying the predictive model to the related interaction data to produce a second set of interaction scores;
identifying, by the processor, a plurality of sub-populations among the second set of interaction scores by applying the clustering model to the second set of interaction scores;
measuring, by the processor, a distance between each of the plurality of sub-populations among the second set of interaction scores;
comparing, by the processor, the distances of the first period of time and the distances of the second period of time; and
generating, by the processor, an alert when the comparison exceeds a predefined threshold.

2. The method as in claim 1, wherein the clustering model is a gaussian mixture model; and
wherein the sub-populations are gaussians.

3. The method as in claim 2, wherein the measured distance is the distance between means of the gaussians.

4. The method as in claim 2, wherein the measured distance is a KL-divergence between a lower gaussian and a upper gaussian; and wherein a mean of each gaussian is used to identify the lower gaussian and the upper gaussian.

5. The method as in claim 1, wherein the predictive model is one of an SVM-based model, a deep learning model, a neural network-based model, a logistic regression model, a linear regression model, a nearest neighbor model, a decision tree model, a PCA-based model, a naive Bayes classifier model, and a k-means clustering model.

6. The method as in claim 1, wherein each interaction is represented by a unique identifier; and wherein a plurality of unique identifiers is randomly selected when extracting the random samples.

7. The method as in claim 1, wherein an interaction comprises data relating to an interaction between a customer and call center in a communication channel.

8. The method as in claim 7, wherein a communication channel is one of automatically transcribed content of a voice conversation, e-mail, chat, and text message.

9. A system for providing unsupervised model health monitoring, comprising:
a computer having a processor and a memory, and
one or more code sets stored in the memory and executed by the processor, which, when executed, configured the processor to:
extract from an interaction database, a first random sample of interaction data relating to a first set of interactions during a first period of time;
score each interaction of the first set of interactions by applying a predictive model to the related interaction data to produce a first set of interaction scores;
identify a plurality of sub-populations among the first set of interaction scores by applying a clustering model to the first set of interaction scores;
measure a distance between each of the plurality of sub-populations among the first set of interaction scores;
extract from the interaction database, a second random sample of interaction data relating to a second set of interactions;
score each interaction of the second set of interactions by applying the predictive model to the related interaction data to produce a second set of interaction scores;
identify a plurality of sub-populations among the second set of interaction scores by applying the clustering model to the second set of interaction scores;
measure a distance between each of the plurality of sub-populations among the second set of interaction scores;
compare the distances of the first period of time and the distances of the second period of time; and
generate an alert when the comparison exceeds a predefined threshold.

10. The system as in claim 9, wherein the clustering model is a gaussian mixture model; and wherein the sub-populations are gaussians.

11. The system as in claim 10, wherein the measured distance is the distance between means of the gaussians.

12. The system as in claim 10, wherein the measured distance is a KL-divergence between a lower gaussian and an upper gaussian; and wherein a mean of each gaussian is used to identify the lower gaussian and the upper gaussian.

13. The system as in claim 9, wherein the predictive model is one of an SVM-based model, a deep learning model, a neural network-based model, a logistic regression model, a linear regression model, a nearest neighbor model, a decision tree model, a PCA-based model, a naive Bayes classifier model, and a k-means clustering model.

14. The system as in claim 9, wherein each interaction is represented by a unique identifier; and wherein a plurality of unique identifiers is randomly selected when extracting the random samples.

15. The system as in claim 9, wherein an interaction comprises data relating to an interaction between a customer and call center in a communication channel.

16. The system as in claim 15, wherein a communication channel is one of automatically transcribed content of a voice conversation, e-mail, chat, and text message.

17. A method for providing unsupervised model health monitoring, performed on a computer having a processor, a memory, and one or more code sets stored in the memory and executed by the processor, the method comprising:
extracting, by the processor, from an interaction database, a plurality of random samples of interaction data relating to respective sets of interactions during a plurality of periods of time;
scoring, by the processor, each interaction of each respective set of interactions by applying a predictive model to the related interaction data to produce a set of interaction scores for each respective set;
identifying, by the processor, a plurality of sub-populations among the each respective set of interaction scores by applying a clustering model to each respective set of interaction scores;
measuring, by the processor, a respective distance between each of the plurality of sub-populations among each of the respective sets of interaction scores;
comparing, by the processor, the respective distances of each period of time; and
generating, by the processor, an alert when a comparison exceeds a predefined threshold for at least one of the respective comparisons.

18. The method as in claim 17, wherein the clustering model is a gaussian mixture model; and wherein the sub-populations are gaussians.

19. The method as in claim 18, wherein the measured distance is the distance between means of the gaussians.

20. The method as in claim 18, wherein the measured distance is a KL-divergence between a lower gaussian and an upper gaussian; and wherein a mean of each gaussian is used to identify the lower gaussian and the upper gaussian.
